(19) United States
(12) Patent Application Publication
(10) Pub. No.: US 2022/0104724 A1
(43) Pub. Date: Apr. 7, 2022

(54) COMPUTER-BASED DYNAMIC RATING OF ATAXIC BREATHING

(71) Applicant: UNIVERSITY OF UTAH RESEARCH FOUNDATION, Salt Lake City, UT (US)

(72) Inventors: Lara M. Brewer CATES, Salt Lake City, UT (US); Robert J. FARNEY, Salt Lake City, UT (US); Sean C. ERMER, Edwards, CO (US); Joseph A. ORR, Park City, UT (US)

(21) Appl. No.: 17/419,133

(22) PCT Filed: Jan. 7, 2020
(86) PCT No.: PCT/US2020/012507

§ 371 Date: Jun. 28, 2021

Related U.S. Application Data
(60) Provisional application No. 62/789,389, filed on Jan. 7, 2019.

(51) Int. Cl. A61B 5/08; A61B 5/00; G16H 50/30; G16H 50/20

(52) U.S. Cl. CPC A61B 5/0816; A61B 5/7264; A61B 5/743; A61B 5/6819; A61B 2562/0247; A61B 5/7278; G16H 50/30; G16H 50/20; A61B 5/6823

(57) ABSTRACT

A computer system receives sensor measurements from a respiration sensor. The computer system generates a particular set of data features from the sensor measurements. The computer system then processes the particular set of data features through a computer-based ataxic breathing rating algorithm. The computer system displays on a user interface an ataxic breathing rating that is calculated from the computer-based ataxic breathing rating algorithm.

### CROSS-REFERENCE TO RELATED APPLICATIONS

This application claims the benefit of and priority to U.S. Provisional Patent Application Ser. No. 62/789,389 filed on Jan. 7, 2019 and entitled “COMPUTER-BASED DYNAMIC RATING OF ATAXIC BREATHING,” which application is expressly incorporated to herein by reference in its entirety.

### GOVERNMENT RIGHTS

This invention was made with government support under Grant Number NNX15A124H awarded by National Aeronautics and Space Administration. The government has certain rights in the invention.

### BACKGROUND

Computers and computing systems have affected nearly every aspect of modern living. Computers are generally involved in work, recreation, healthcare, transportation, entertainment, household management, etc. Computers have made tremendous advancements within the field of medical treatment and diagnosis. The greater inclusion of computer-aided medical treatment has led to diagnosis accuracy that often exceeds that of long-time practitioners. In many cases, computer-based technologies are able to leverage sensor data and apply calculations that are not possible for humans.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Disclosed embodiments include a computer system that receives sensor measurements from a respiration sensor. The computer system generates a particular set of data features from the sensor measurements. The computer system then processes the particular set of data features through a computer-based ataxic breathing rating algorithm. The computer system displays on a user interface an ataxic breathing rating that is calculated from the computer-based ataxic breathing rating algorithm.

Disclosed embodiments also comprise a computer-implemented method for computer-based dynamic rating of ataxic breathing severity. The computer-implemented method is executed on one or more processors using instructions stored in memory. The computer-implemented method comprises receiving sensor measurements from a respiration sensor. The respiration sensor is configured to be attached to a human user. The one or more processors then identify a subset of breaths within the sensor measurements. The one or more processors generate an interbreath interval standard deviation. The interbreath interval standard deviation indicates a standard deviation between interbreath intervals within the subset of breaths. The one or more processors also generate an interbreath interval Poincaré summation by calculating a sum of Euclidean distances between consecutive Poincaré data points for interbreath intervals within the subset of breaths. The one or more processors execute a support vector machine classifier process. Further, the one or more processors generate an ataxic breathing rating by processing at least the interbreath interval standard deviation and the Poincaré summation within the support vector machine classifier process. Further still, the one or more processors display the ataxic breathing rating on a user interface.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings.

FIG. 1 illustrates an embodiment of a system for ataxic breathing rating.

FIG. 2 illustrates an embodiment of sensor placement on a subject.

FIG. 3 illustrates an embodiment of a stacked bar chart displaying the distribution of scores given by each rater.

FIGS. 4A-4E illustrates charts depicting various sensor measurements.

FIG. 5 illustrates a flowchart of an embodiment of a method for ataxic breathing rating.

FIG. 6 illustrates a flowchart of another embodiment of a method for ataxic breathing rating.

### DETAILED DESCRIPTION

Disclosed embodiments provide a way of monitoring, in real-time, respiratory patterns that may predict unexpected respiratory complications including death due to opioid toxicity. Typically, ataxic breathing is seen in patients receiving opioids but heretofore there has been no way to use this information clinically. This inability to utilize ataxic breathing as a warning sign has been due at least in part to the lack of technology to identify ataxic breathing in real-time. Instead, conventionally ataxic breathing patterns are identified after the fact by specialists looking over breathing readings from a patient that was previously being monitored.

Disclosed embodiments provide a method, employing a Poincaré return graph, to to accurately identify these patterns. Disclosed embodiments also comprise a scale that corresponds with opioid toxicity. In at least one embodiment, the scale of 3-4 indicates potentially lethal toxicity. One will appreciate that the actual values of the scale are somewhat arbitrary but instead the ability to map ataxic breathing to a scale in real-time provides significant technical benefits to the field. Embodiments could be applied in the perioperative environment to monitor patients and prevent unexpected respiratory arrests. Further embodiments may include analysis of patients undergoing sleep studies in order to predict unexpected high risks for opioids that are used chronically.

Opioid induced respiratory depression is traditionally recognized by assessment of respiratory rate, hemoglobin oxygen saturation, end-tidal CO2, and mental status. Although an irregular or ataxic breathing pattern is widely recognized as a manifestation of opioid effects, the presence of ataxic breathing is not monitored or scored in real-time. A major obstacle to widespread monitoring for ataxic breathing is the necessity for manual offline analysis. In at least one embodiment, an automated machine learning algorithm is used to quantify the severity in ataxic breathing pattern in real-time for patients experiencing drug-induced respiratory depression.

A variety of means exist to assess the respiratory status of patients receiving opioids in the perioperative period; however no universal, codified standard of care currently exists. Typically, a health care professional will enter the patient's room and perform periodic manual assessments, or “spot checks” every several hours, during which respiratory rate, hemoglobin oxygen saturation (SpO2), end-tidal CO2, and mental status may be observed. The primary limitation surrounding non-continuous monitoring is that patients can become alert when interacting with a healthcare professional that is assessing their status. Because of this, and the fact that apneic periods can be cyclical in nature, these types of assessments can miss critical signs regarding patient status.

For continuous monitoring, oxygen saturation (SpO2) and respiratory rate are the vital signs most often measured. For patients receiving supplemental oxygen (which is often prescribed to patients receiving opioid analgesic pain care), SpO2 can take up to 3 minutes to identify the onset of apnea or hypoventilation. A measurement of respiratory rate can alleviate this limitation by identifying the decreased ventilation earlier, however this has its own drawbacks. One limitation is that apneas which are cyclical in nature may be missed by a practitioner who is responding to a respiratory rate alarm. Additionally, though respiratory rate is typically displayed as “breaths per minute” the calculation can be performed in a number of ways. For example, one could count the total number of breaths in a sixty second window, average the respiratory rate observed for a fixed number of recent breaths or minutes, extrapolate the respiratory rate from the time between the last two breaths, or combine these two methods in some manner. Because interbreath intervals can be highly variable, one may draw the wrong conclusion when asked to interpret a respiratory rate. For example, a patient who is apneic for 30 seconds and then breathes every 5 seconds could display the same respiratory rate as a patient who breathes once every 10 seconds, despite the difference in regularity observed.

Although an irregular or ataxic breathing pattern is widely recognized as a manifestation of opioid effects, the presence of ataxic breathing is not routinely monitored or scored. A major obstacle to widespread monitoring for ataxic breathing is the necessity for manual offline analysis.

In the perioperative environment, commonly monitored parameters include arterial oxygen saturation by pulse oximetry (SpO2), respiratory rate, end-tidal carbon dioxide (PₑₜCO₂.) by capnometry, and systematic sedation assessments. The effectiveness of oximetry monitoring is limited because, 1) It is insensitive to changes in ventilation especially during the routine use of supplemental oxygen; 2) Criteria for defining respiratory failure are not standardized (e.g. the threshold SpO2 level such as 85% or 90%); and, 3) Periodic assessments, or “spot checks”, rather than continuous monitoring are inaccurate and lead to patient arousal during the physical assessment. Reduced breath rate (typically <8-10 breaths per minute) is the most widely used single clinical parameter to identify opioid induced respiratory depression. However, the measurement of ventilatory frequency by physical examination is often neglected or inaccurate, and detection of respiratory rate by other technological methods is unreliable for bradypnea. Monitoring of PₑₜCO₂ is a more direct way of assessing ventilation. However, capnometry is expensive and may be difficult to interpret in a non-intubated patient. Finally, systematic sedation assessments may be misleading since the patient is aroused during assessment, but may then return to a dangerous state after the clinician leaves the bedside. In some cases, severe respiratory depression has been observed in patients with minimal sedation.

Although widely recognized as a manifestation of opioid's toxic effects on the central respiratory pattern generator, irregular or ataxic breathing is not easy to quantify especially in real-time. Accordingly, the degree of ataxic breathing is not routinely monitored. Real-time quantification of ataxic breathing severity may add important information to the conventionally monitored parameters of SpO₂, breath rate, and PₑₜCO₂.

In at least one embodiment, the disclosed invention can change patient care. For example, a monitor which displays an ataxic breathing score would provide the healthcare practitioner with more complete information regarding the respiratory status of the patient and aid them in making a more informed decision. Ataxic breathing severity scoring could be adjusted to highlight problems over a much more dynamic time scale than respiratory rate, which is simply interpreted as the number of breaths in a minute, and additionally could help identify patients who are experiencing cyclical periods of apnea.

Additionally, this score may be able to provide an early indicator of patient sensitivity to opioids before the patient begins experiencing severe, adverse respiratory events. The degree to which one's breathing variability is affected by opioids may correlate with dosing and help prescribe proper treatment. Currently, additional data is required regarding this hypothesis. Additionally, outpatients may be sent home with a monitor which can assess ataxic breathing status and record that information for a physician. Similarly to the above, this could be used to help prescribe future treatment, or alert family or caretakers if a dangerous pattern of ataxic breathing begins to manifest.

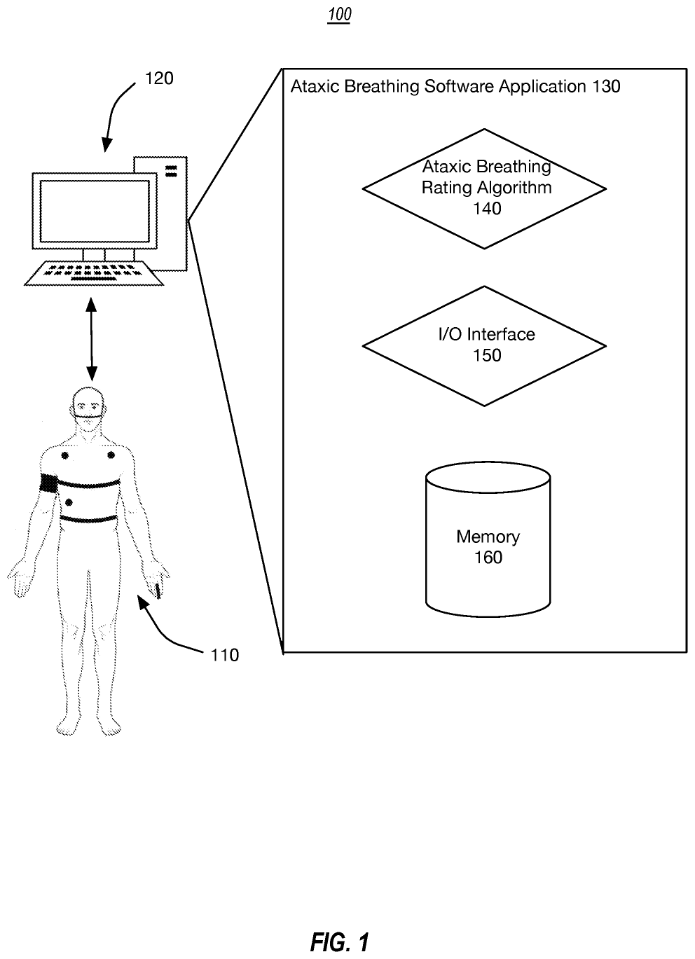

FIG. 1 depicts an embodiment of a system 100 for ataxic breathing rating. The depicted system 100 is provided for the sake of example and explanation. One will appreciate that the system 100 may be otherwise configured or described and still fall within the present description of the invention.

The system 100 for ataxic breathing rating comprise one or more sensors attached to a patient 110. The sensors provide sensor measurements to a computer system 120 that is executing an ataxic breathing software application 130. The ataxic breathing software application 130 comprises an ataxic breathing rating algorithm 140, an I/O (Input/Output) Interface 150, and memory 160. As will be described more fully below, the depicted components operate together to analyze sensor measurements and in near real-time generate ataxic breathing ratings.

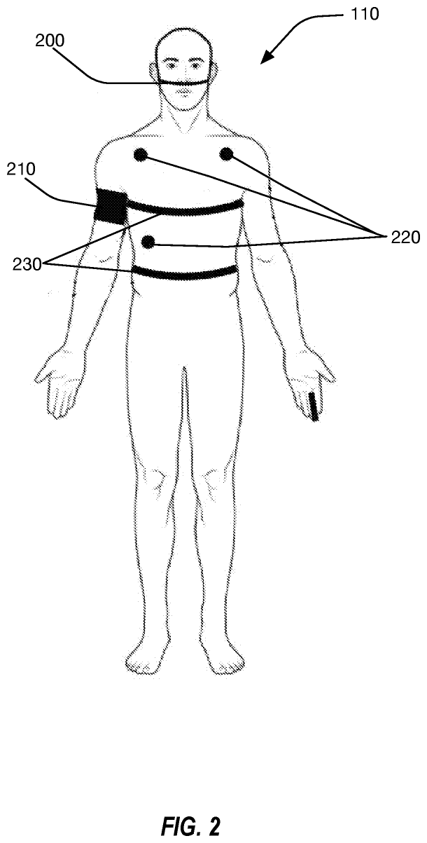

In at least one embodiment, the system 100 receives sensor measurements from a respiration sensor attached to a patient 110. FIG. 2 illustrates an embodiment of sensor placement on a patient 110. The depicted sensors comprise a nasal cannula 200 that includes an intranasal pressure transducer and a capnometer, a blood pressure cuff 210, transpulmonary electrical impedance leads 220, and respiratory inductance plethysmography (RIP) bands 230. The Respiratory Inductance Plethysmography (RIP) bands may comprise a chest band sensor and an abdominal band sensor. One will appreciate that the depicted sensors and placements are shown for the sake of example and explanation. In various embodiments, each depicted sensor may be used independently or in combination with the other depicted sensors or other capable sensors that are not depicted may be utilized.

Once the system 100 receives the sensor measurements, the computer system 120 processes the sensor measurements within the ataxic breathing software application 130. Specifically, the computer system 120 generates a particular set of data features from the sensor measurements. When generating the particular set of data features, the computer system 120 may first identify a subset of breaths within the sensor measurements, such as a subset of 30 breaths, a subset of 90 breaths, or any other subset of interest. The particular set of data feature may comprise one or more of an interbreath interval standard deviation, an interbreath interval Poincaré summation, a tidal volume standard deviation, or a tidal volume Poincaré summation.

The ataxic breathing software application 130 then processes the particular set of data features through a computer-based ataxic breathing rating algorithm 140. In at least one embodiment, the ataxic breathing rating algorithm 140 comprises a support vector machine classifier process. Further, in at least one embodiment the computer-based ataxic breathing rating algorithm 140 generates an ataxic breathing rating by processing at least the interbreath interval standard deviation and the Poincaré summation within the support vector machine classifier process. One will appreciate that an ataxic breathing rating may comprise a machine learning approach; however such an approach is not necessary in a final implementation.

Once the ataxic breathing rating is calculated, the I/O Interface displays the rating (and optionally the Poincaré plot) on a user interface in near-real time. As such, a health care provider is able to readily identify data about a patient 110 in real-time. One will appreciate that using conventional processes, an ataxic breathing rating would not otherwise be available until breathing data was manually analyzed at some later time.

In at least one embodiment, an ataxic breathing rating comprises respiratory inductance plethysmography and nasal pressure as two possible signals to calculate the score. However, the similarity in results obtained between these two signals indicate that any signal which provides accurate, real-time breath marks can likely be used to calculate the ataxic breathing score. These may include any motion or airflow sensor such as accelerometry, impedance or capnography, or be further expanded to signals such as photoplethysmography which is capable of identifying respiration in addition to blood oxygen saturation.

The ataxic breathing rating is a measure of “variability” in the respiratory pattern made evident via Poincaré return graph analysis. The data features of interest may comprise statistical calculations of the sensor measurements such as the standard deviation in the interbreath intervals and other measures such as signal entropy. Other statistics could be used to identify the extent of variability in the timing of subsequent breaths and variability in tidal volume of the respiration signal over a moving window of monitored breaths. While several data features may be used, a final implementation of the score may include only a single calculation (i.e. entropy of the interbreath intervals in an epoch of breath data) rather than a machine learning approach. In contrast, in at least one embodiment, the system 100 utilizes a sum of Euclidean distances between consecutive points in a Poincaré plot for a subset of breaths, which is described further below. The subset of breaths may be defined as a number of different measurements, such as 30 breaths per analysis, or as few as 4 and as many as 90 breaths.

In at least one embodiment, upon receiving the sensor measurements, such as Intranasal pressure and RIP waveforms, the computer system 120 filters the sensor measurements using a zero phase first-order low pass filter. For intranasal pressure and RIP waveform data, breath marks were identified. The interbreath interval (time in seconds between subsequent breaths) is calculated for each breath mark.

A tidal volume factor change based on intranasal pressure was obtained by 1) taking the square root of the pressure signal, 2) integrating the signal, 3) multiplying by the scaling factor of 2000, 4) measuring the peak to trough height at each breath to obtain tidal volume, and 5) calculating the absolute value of the factor change in tidal volume from one breath to the next throughout the data set using the following equation:

$$Factor Change in {V}_{T} = {e}^{\vert ln ( \frac{{V}_{{T}_{i}}}{{V}_{{T}_{i + 1}}} ) \vert} ,$$

where V_{T} is tidal volume (mL) measured in step four above as the peak to trough height and “i” is the breath number in the epoch.

The tidal volume factor change based on respiratory inductance plethysmography bands is obtained by 1) summing the chest and abdomen signals, 2) applying a linear transformation (y=mx+b) to convert the signal to tidal volume, 3) measuring the peak to trough height at each breath to obtain tidal volume, and 4) calculating the absolute value of the factor change in tidal volume from one breath to the next throughout the data set.

Turning now to the ataxic breathing rating algorithm 140, in at least one embodiment, the ataxic breathing rating algorithm 140 comprises a Support Vector Machine (SVM) classifier to perform multiclass classification. The SVM may comprise a fine Gaussian kernel function, ordinal classification, and five-fold cross validation. The labels used for learning may comprise a rounded average of the domain experts' scores of ataxic breathing.

The inputs to the SVM classifier may comprise a combination of the following: 1) the standard deviation of all twenty-nine interbreath intervals (also referred to herein as the “interbreath interval standard deviation”), 2) the standard deviation of tidal volumes for all twenty-nine subsequent breaths, 3) the Sum of Euclidean distances between consecutive Poincaré data points for interbreath intervals (also referred to herein as “interbreath interval Poincaré summation”), and 4) the Sum of Euclidean distances between consecutive Poincaré data points for tidal volume factor change. In at least one embodiment, the inputs comprise only the interbreath interval standard deviation and the interbreath interval Poincaré summation.

The Sum of Euclidean Distances for Interbreath Interval may be calculated as

Σᵢ₌₁^{N-2}\sqrt{(IBIᵢ−IBIᵢ₊₁)²+(IBIᵢ₊₁−IBIᵢ₊₂)²},

where IBI is the interbreath interval and “i” is the breath number.

The Sum of Euclidean Distances for Tidal Volume Factor Change may be calculated as

$${\sum }_{i = 1}^{N - 2} \sqrt{{( \frac{{TV}_{i} - {TV}_{i + 1}}{TVi} )}^{2} + {( \frac{{TV}_{i + 1} - {TV}_{i + 2}}{TVi + 1} )}^{2}} ,$$

where TV indicates the tidal volume of each breath and “i” is the breath number.

In at least one embodiment, training the SVM classifier comprises utilizing data segments that are separated into training and test sets. To ensure no single subject existed in both data sets, subjects were randomly added to the training set until at least 50% of the data is included. The remaining segments were then used as the test set. The four training features are then input to a support vector machine (SVM) classifier.

Classifier training may be performed two times: 1) using features derived from the chest and abdomen RIP band sum waveforms and 2) using features derived from intranasal pressure waveforms.

In order to account for bias that may occur due to subject sampling during classifier training, the training set selection and SVM training processes may be repeated 1,000 times. The final SVM model in each iteration was exported and used to classify ataxic breathing severity for each of the 1,000 randomized test sets.

As explained above, in at least one embodiment, the ataxic breathing rating algorithm may utilize a set of four data features from the sensor measurements (standard deviation of interbreath intervals, the average breath-by-breath tidal volume factor change, and Euclidean distance between consecutive Poincaré data points for both interbreath interval, and breath-by-breath tidal volume factor change). The computer system 120 may also utilize an expanded set of data features from the sensor measurements to train the SVM. For example, six data features that may also be used in combination or separately include: 1) average signal entropy for all interbreath intervals; 2) average signal entropy for tidal volume factor changes; 3) area of the convex hull which encompasses the twenty-nine points of the Poincaré plot for interbreath intervals; 4) area of the convex hull which encompasses the twenty-nine points of the Poincaré plot for tidal volume factor changes; 5) Maximum Euclidean distance calculated on a subset of five interbreath intervals; and 6) Maximum Euclidean distance calculated on a subset of five tidal volume factor changes. For the final two data features, all subsets of five adjacent data points are calculated, and the maximum Euclidean distance of all subsets is used as the final value. The learning process may be repeated multiples times (e.g., 1,000 times) using a training set of at least 50% of the data for training in each iteration as described above.

An experiment was performed to at least in part validate the function of an embodiment of the present invention. In the experiment, two outcome measures are defined: Krippendorff s Alpha, and Vanbelle's Kappa. The primary outcome measure is Krippendorff's Alpha, which calculates interrater reliability for N raters on an ordinal data set. Values of Krippendorff's Alpha range from 0 to 1, where an alpha of 1 indicates perfect agreement between raters and an alpha of 0 indicates all agreement is due to random chance. For Krippendorff's Alpha, an ordinal rating scale was utilized. The inputs for this metric were the individual domain expert's scores and the SVM classifier's scores.

Vanbelle's Kappa expands on Krippendorff's Alpha by measuring the proportion of agreement between an isolated rater and a group of raters. It corrects for the level of agreement that may occur by chance and additionally considers scores from multiple raters. The range of this statistic is 0 to 1, where 1 indicates perfect agreement and 0 indicates all agreement is due to random chance. For Vanbelle's Kappa, each of the individual domain expert's scores or the SVM classifier's scores is compared to the average of the other three scores.

The acceptance criteria for both variables was set at alpha≥0.8 as has been suggested by Krippendorff. The average and the 2.5 and 97.5 percentiles of data across all 1,000 iterations were calculated to give the mean and approximate 95% confidence intervals of the data.

Subject characteristics are summarized in Table 1.

TABLE

| | Age (years) | | | Height (cm) | | | Weight (kg) | | | BMI (kg/m²) | | |
|---|---|---|---|---|---|---|---|---|---|---|---|---|
| Metric | All | M | F | All | M | F | All | M | F | All | M | F |
| Mean (SD) | 28 (7) | 27 (6) | 28 (7) | 175 (9) | 182 (6) | 168 (5) | 77 (14) | 84 (15) | 70 (9) | 25 (4) | 26 (4) | 25 (3) |
| Range | 18-41 | 18-39 | 19-41 | 160-193 | 167-193 | 160-175 | 56-103 | 61-103 | 56-88 | 18-31 | 18-31 | 21-31 |

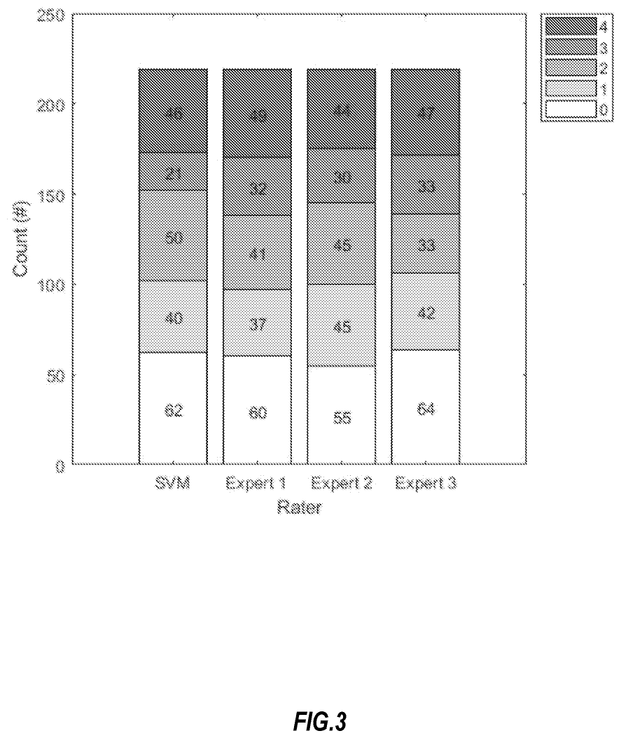

Each of three domain experts evaluated 219 30-breath data epochs sampled from all 26 subjects. FIG. 3 depicts a stacked bar plot which details the count of each score assigned by each rater for the full set of 219 data epochs. In the case of the support vector machine, the values for each portion of the bar were obtained by calculating the mode score assigned to each 30-breath epoch by the support vector machine over 1000 iterations.

At least 100 data segments were chosen as training sets for each of the 1,000 iterations of the SVM classifier learning process, with 105 data segments being chosen on average. The interrater reliability results for the remaining 114 data segments (the test sets) using features based on the RIP band signal are presented in Table 2. The interrater reliability results for the remaining 114 data segments (the test sets) using features based on the intranasal pressure signal are presented in Table 3.

Table 2 shows interrater reliability analysis using respiratory inductance plethysmography (RIP) bands as the source for interbreath interval and tidal volume factor change parameters. The Basic Classifier was trained on the original set of 4 features. The Expanded Classifier was trained on an expanded set of ten features. Kn is reported for the SVM classifier compared to the domain experts.

TABLE 2

| | Basic Classifier | Expanded Classifier |
|---|---|---|
| | (4 RIP | (10 RIP |
| | Band Features) | Band Features) |
| Statistic | Mean (95% CI) | Mean (95% CI) |
| Krippendorff's alpha | 0.912 (0.852-0.949) | 0.905 (0.854-0.947) |
| Vanbelle's Kappa | 0.970 (0.951-0.983) | 0.976 (0.961-0.987) |

Table 3 shows interrater reliability analysis using intranasal pressure as the source for interbreath interval and tidal volume factor change parameters. The Basic Classifier was trained on the original set of 4 features. The Expanded Classifier was trained on an expanded set of ten features. Vanbelle's Kappa is reported for the SVM classifier compared to the domain experts.

TABLE 3

| | Basic Classifier | Expanded Classifier |
|---|---|---|
| | (4 Intranasal | (10 Intranasal |
| | Pressure Features) | Pressure Features) |
| Statistic | Mean (95% CI) | Mean (95% CI) |
| Krippendorff's alpha | 0.899 (0.819-0.941) | 0.893 (0.813-0.936) |
| Kₙ | 0.961 (0.921-0.979) | 0.959 (0.923-0.978) |

Per the results for the tested at least one embodiment, the Support Vector Machine classifier and the domain experts are in agreement (Krippendorff's Alpha>0.8 and Vanbelle's Kappa>0.8) in their assessment of ataxic breathing severity. Krippendorff s Alpha and Vanbelle's Kappa showed average alpha values>0.8, the acceptance criteria, for 1,000 iterations of training. The highest alpha values were observed with the ten-feature RIP band-based classifier, with a Krippendorff s Alpha value of 0.905 and a Vanbelle's Kappa value of 0.976.

Previous investigations have assessed the effects of acute opioid administration on interbreath intervals and flow characteristics but have not specifically analyzed the breathing patterns for ataxia. Erratic or ataxic breathing has been found in a high proportion of patients using chronic opioids. Convectional characterization of ataxic breathing is by variable respiratory rate and tidal volumes with pauses that do not seem to have any clear periodicity. When extreme, the pattern is characterized by cluster breathing in which breaths occur episodically in bursts separated by long pauses. Severe ataxic breathing is obvious by visual inspection of the respiratory flow signal; however, minimal or intermediate abnormalities may be difficult to identify with certainty. Until now, the diagnosis and quantification of ataxic breathing due to opioids has been subjective.

Despite wide spread recognition that irregular breathing is a frequently observed manifestation of opioid toxicity, there has been no practical system to capture this property for investigational or clinical applications. The selected machine learning algorithm, a support vector machine classifier, showed high agreement with a panel of domain experts who examined the breathing patterns after the fact. Regardless of the number of features or whether the respiration was monitored by the most basic technology (i.e. intranasal pressure), the interrater agreement was higher than 0.893 for all alpha values assessed. Interestingly, results based on features from intranasal pressure and Respiratory Inductive Plethysmography (RIP band signals) were not significantly different. Additionally, results with tidal volume features removed were not significantly different from those with tidal volume included. This indicates that any signal with adequate breath detection capabilities could theoretically be used to evaluate ataxia. Overall, the experiment confirmed that the SVM reliably quantifies typical opioid induced ataxic breathing patterns in a manner consistent with a panel of domain experts.

Although the variations in respiratory rate, tidal volume and rhythm secondary to opioid administration visually appear to be random or stochastic, the irregular breathing pattern has been explained as a manifestation of quantal slowing. Episodic or cluster breathing, characterized by brief periods of continuous breathing interspersed with periods of apnea, has been observed in species of all classes of vertebrates, for example when respiratory drive is reduced, with hypothermia, commencing hibernation or during certain developmental phases. At least one embodiment utilizes a modified Poincaré plot based on the interbreath intervals to also reflect the variability of tidal volume. These plots significantly enhanced the visual recognition of stable versus increasingly variable breathing patterns.

In summary, we confirmed that a machine learning algorithm classified typical ataxic breathing patterns associated with variable degrees of opioid toxicity in a manner consistent with domain experts.

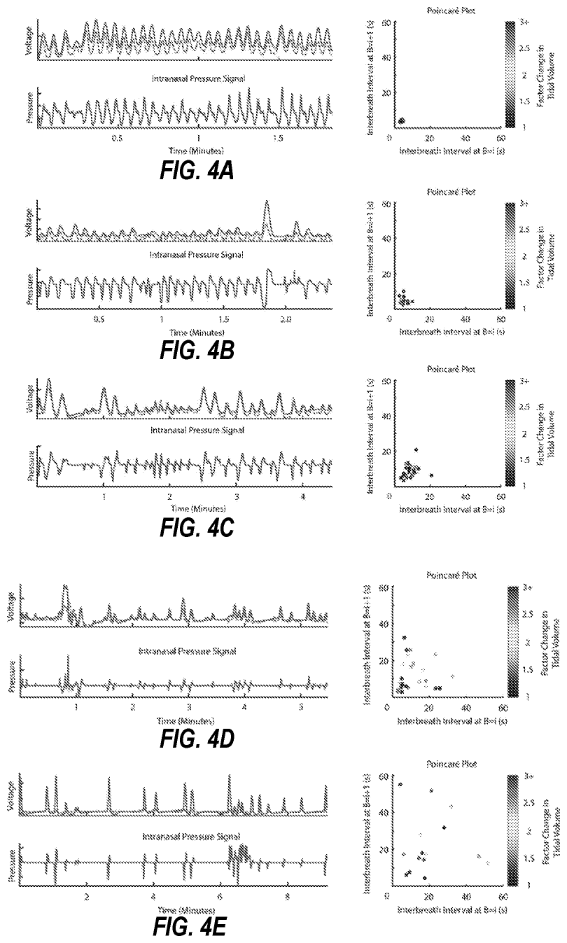

FIGS. 4A-4D illustrates charts depicting various sensor measurements. Each panel contains two waveforms (left), respiratory inductance plethysmography (RIP) and intranasal pressure, and a matching Poincaré plot (right) for interbreath intervals (sec) from subsequent breaths (B=i and B=i+1). Each point in the Poincaré plot represents the absolute value for factor change in tidal volume between subsequent breaths at B=i and B=i+1. For example a factor change of 2 indicates the tidal volume either doubled or halved. FIG. 4A represents an ataxic breathing to rating of 0 (no ataxia). FIG. 4B represents an ataxic breathing rating of 1 (mild ataxia). FIG. 4C represents an ataxic breathing rating of 2 (moderate ataxia). FIG. 4D represents an ataxic breathing rating of 3 (severe ataxia). FIG. 4E represents an ataxic breathing rating of 4 (severe ataxia+cluster breathing).

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

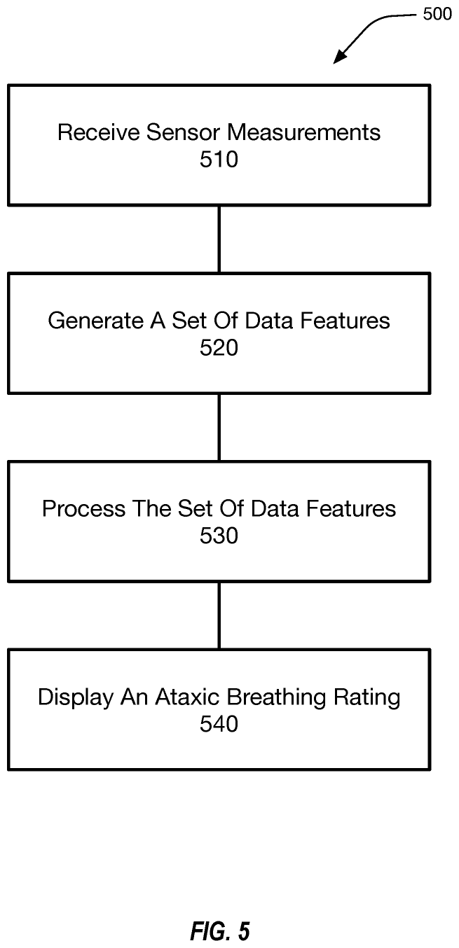

FIG. 5 illustrates a flowchart of an embodiment of a method 500 for ataxic breathing rating. The method includes an act 510 of receiving sensor measurements. Act 510 comprises receiving sensor measurements from a respiration sensor. For example, as depicted and described with respect to FIG. 1 and FIG. 2, a computer system 120 receives sensor measurements from a patient 110 wearing one or more respiration sensors 200, 210, 220, 230.

The method 500 also includes an act 520 of generating a set of data features. Act 520 comprises generating a particular set of data features from the sensor measurements. For example, as depicted and described with respect to FIG. 1, the computer system 120 generates data features from the sensor readings. The data features may comprise, among data features, interbreath standard deviation and interbreath interval Poincaré summation.

Additionally, method 500 includes an act 530 of processing the set of data features. Act 530 comprises processing the particular set of data features through a computer-based ataxic breathing rating algorithm. For example, as depicted and described with respect to FIG. 1, an ataxic breathing rating algorithm 140, such as an SVM process, may be utilized to processing the particular set of data features that the computer system 120 derives from the sensor measurements.

Further, method 500 includes an act 540 of displaying an ataxic breathing rating. Act 540 comprises displaying on a user interface an ataxic breathing rating that is calculated from the computer-based ataxic breathing rating algorithm. For example, as depicted with respect to FIG. 1 and FIG. 3, the I/O interface 150 within the ataxic breathing software application 130 can display on the computer system an ataxic breathing rating such as the ratings displayed and described in FIG. 3.

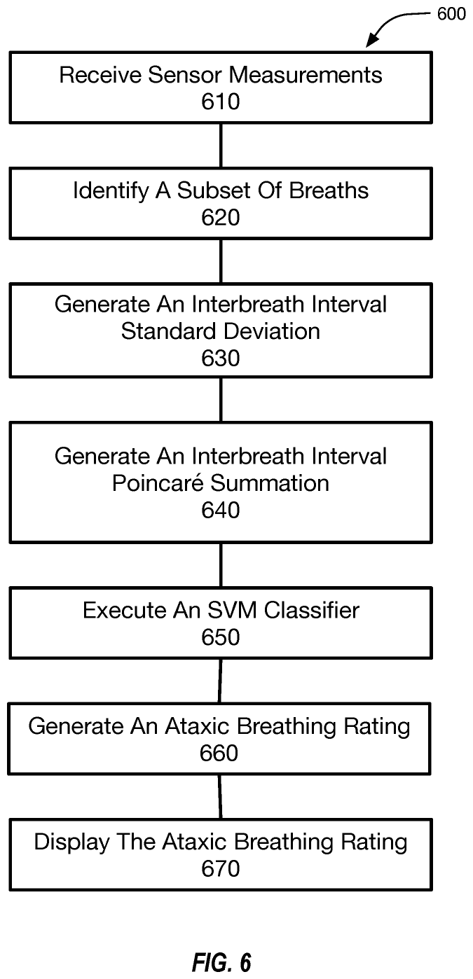

Additional or alternative methods may also be used to identify an ataxic breathing rating. For example, FIG. 6 illustrates a flowchart of another embodiment of a method for ataxic breathing rating. FIG. 6 depicts a method 600 that includes an act 610 of receiving a sensor measurement. Act 610 comprises receiving sensor measurements from a respiration sensor, wherein the respiration sensor is configured to be attached to a human user. For example, as depicted and described with respect to FIG. 1 and FIG. 2, a computer system 120 receives sensor measurements from a patient 110 wearing one or more respiration sensors 200, 210, 220, 230.

Method 600 also includes an act 620 of identifying a subset of breaths. Act 620 comprises identifying a subset of breaths within the sensor measurements. For example, the computer system 120 may identify a particular subset of breaths that comprises 4 breaths, 30 breaths, 90 breaths, breaths over the span of 30 seconds, breaths over the span of a minute, or any other desirable subset of breaths. The subset of breaths are then analyzed as described herein.

Additionally, method 600 includes an act 630 of generating an interbreath interval standard deviation. Act 630 comprises generating an interbreath interval standard deviation, wherein the interbreath interval standard deviation indicates a standard deviation between interbreath intervals within the subset of breaths. For example, the computer system 120 can identify breath intervals using both conventional and novel methods. The computer system can then calculate a standard deviation of the intervals.

Method 600 further includes an act 640 of generating an interbreath interval Poincaré summation. Act 630 comprises generating an interbreath interval Poincaré summation by calculating a sum of Euclidean distances between consecutive Poincaré data points for interbreath intervals within the subset of breaths. For example, the computer system 120 can identify breath intervals using both conventional and novel methods. The computer system can then calculate a Poincaré summation of the intervals using the methods described above.

In addition, method 600 includes an act 650 of executing an SVM classifier. Act 650 comprises executing, on the one or more processors, a support vector machine classifier process For example, as depicted in FIG. 1, the Ataxic Breathing Rating Algorithm 140 may comprise a support vector machine classifier process that has been configured and trained according to the description provided herein.

Further, method 600 includes an act 660 of generating an ataxic breathing rating Act 660 comprises generating an ataxic breathing rating by processing at least the interbreath interval standard deviation and the Poincaré summation within the support vector machine classifier process. For example, as depicted in FIG. 1, the Ataxic Breathing Rating Algorithm 140 generates an ataxic breathing rating based upon the received data features.

Further still, method 600 includes an act 670 of displaying the ataxic breathing rating. Act 670 comprises displaying the ataxic breathing rating on a user interface. For example, as depicted with respect to FIG. 1 and FIG. 3, the I/O interface 150 within the ataxic breathing software application 130 can display on the computer system an ataxic breathing rating such as the ratings displayed and described in FIG. 3.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Computing system functionality can be enhanced by a computing systems' ability to be interconnected to other computing systems via network connections. Network connections may include, but are not limited to, connections via wired or wireless Ethernet, cellular connections, or even computer to computer connections through serial, parallel, USB, or other connections. The connections allow a computing system to access services at other computing systems and to quickly and efficiently receive application data from other computing systems.

Interconnection of computing systems has facilitated distributed computing systems, such as so-called “cloud” computing systems. In this description, “cloud computing” may be systems or resources for enabling ubiquitous, convenient, on-demand network access to a shared pool of configurable computing resources (e.g., networks, servers, storage, applications, services, etc.) that can be provisioned and released with reduced management effort or service provider interaction. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Cloud and remote based service applications are prevalent. Such applications are hosted on public and private remote systems such as clouds and usually offer a set of web based services for communicating back and forth with clients.

Many computers are intended to be used by direct user interaction with the computer. As such, computers have input hardware and software user interfaces to facilitate user interaction. For example, a modern general purpose computer may include a keyboard, mouse, touchpad, camera, etc. for allowing a user to input data into the computer. In addition, various software user interfaces may be available.

Examples of software user interfaces include graphical user interfaces, text command line based user interface, function key or hot key user interfaces, and the like.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A computer system for computer-based dynamic rating of ataxic breathing severity, comprising:
one or more processors; and
one or more computer-readable media having stored thereon executable instructions that when executed by the one or more processors configure the computer system to perform at least the following:
receive sensor measurements from a respiration sensor;
generate a particular set of data features from the sensor measurements;
process the particular set of data features through a computer-based ataxic breathing rating algorithm; and
display on a user interface an ataxic breathing rating that is calculated from the computer-based ataxic breathing rating algorithm.

2. The computer system of claim 1, wherein particular set of data feature comprises an interbreath interval standard deviation.

3. The computer system of claim 2, wherein particular set of data feature comprises an interbreath interval Poincaré summation.

4. The computer system of claim 1, wherein the computer-based ataxic breathing rating algorithm comprises a support vector machine classifier process.

5. The computer system of claim 4, wherein the executable instructions include instructions that are executable to configure the computer system to:
generate an interbreath interval standard deviation, wherein the interbreath interval standard deviation indicates a standard deviation between interbreath intervals within a subset of breaths;
generate an interbreath interval Poincaré summation by calculating a sum of Euclidean distances between consecutive Poincaré data points for interbreath intervals within the subset of breaths;
execute, on the one or more processors, a support vector machine classifier process; and
generate an ataxic breathing rating by processing at least the interbreath interval standard deviation and the interbreath interval Poincaré summation within the support vector machine classifier process.

6. The computer system of claim 1, wherein the executable instructions include instructions that are executable to configure the computer system to:
display on a user interface a corresponding Poincaré plot that is calculated from the computer-based ataxic breathing rating algorithm.

7. The computer system of claim 1, wherein the respiration sensor comprises an intranasal pressure sensor.

8. A computer system for computer-based dynamic rating of ataxic breathing severity, comprising:
one or more processors; and
one or more computer-readable media having stored thereon executable instructions that when executed by the one or more processors configure the computer system to perform at least the following:
receive sensor measurements from a respiration sensor, wherein the respiration sensor is configured to be attached to a human user;
identify a subset of breaths within the sensor measurements;
generate an interbreath interval standard deviation, wherein the interbreath interval standard deviation indicates a standard deviation between interbreath intervals within the subset of breaths;
generate an interbreath interval Poincaré summation by calculating a sum of Euclidean distances between consecutive Poincaré data points for interbreath intervals within the subset of breaths;
execute, on the one or more processors, a support vector machine classifier process;
generate an ataxic breathing rating by processing at least the interbreath interval standard deviation and the interbreath interval Poincaré summation within the support vector machine classifier process; and
display the ataxic breathing rating on a user interface.

9. The computer system of claim 8, wherein the executable instructions include instructions that are executable to configure the computer system to generate a tidal volume standard deviation, wherein the tidal volume standard deviation indicates a standard deviation between tidal volumes within the subset of breaths.

10. The computer system of claim 9, wherein the executable instructions include instructions that are executable to configure the computer system to generate an tidal volume Poincaré summation by calculating a sum of Euclidean distances between consecutive Poincaré data points for tidal volume change within the subset of breaths.

11. The computer system of claim 10, wherein generating an ataxic breathing rating further comprises processing at least the tidal volume standard deviation, the tidal volume Poincaré summation, the interbreath interval standard deviation, and the interbreath interval Poincaré summation within the support vector machine classifier process.

12. The computer system of claim 8, wherein the respiration sensor comprises a chest band sensor.

13. The computer system of claim 8, wherein the respiration sensor comprises an intranasal pressure sensor.

14. A computer-implemented method for computer-based dynamic rating of ataxic breathing severity, the computer-implemented method executed on one or more processors using instructions stored in memory, the computer-implemented method comprising:
receiving sensor measurements from a respiration sensor, wherein the respiration sensor is configured to be attached to a human user;
identifying a subset of breaths within the sensor measurements;
generating an interbreath interval standard deviation, wherein the interbreath interval standard deviation indicates a standard deviation between interbreath intervals within the subset of breaths;
generating an interbreath interval Poincaré summation by calculating a sum of Euclidean distances between consecutive Poincaré data points for interbreath intervals within the subset of breaths;
executing, on the one or more processors, a support vector machine classifier process;
generating an ataxic breathing rating by processing at least the interbreath interval standard deviation and the interbreath interval Poincaré summation within the support vector machine classifier process; and
displaying the ataxic breathing rating on a user interface.

15. The computer-implemented method of claim 14, further comprising generating a tidal volume standard deviation, wherein the tidal volume standard deviation indicates a standard deviation between tidal volumes within the subset of breaths.

16. The computer-implemented method of claim 15, wherein generating an ataxic breathing rating further comprises processing at least the tidal volume standard deviation, the interbreath interval standard deviation, and the interbreath interval Poincaré summation within the support vector machine classifier process.

17. The computer-implemented method of claim 14, further comprising generating a tidal volume Poincaré summation by calculating a sum of Euclidean distances between consecutive Poincaré data points for tidal volume change within the subset of breaths.

18. The computer-implemented method of claim 17, wherein generating an ataxic breathing rating further comprises processing the tidal volume Poincaré summation, the interbreath interval standard deviation, and the interbreath interval Poincaré summation within the support vector machine classifier process.

19. The computer-implemented method of claim 14, wherein the respiration sensor comprises a chest band sensor.

20. The computer-implemented method of claim 14, wherein the respiration sensor comprises an intranasal pressure sensor.
